(19) United States
(12) Patent Application Publication
(10) Pub. No.: US 2006/0253014 A1
(43) Pub. Date: Nov. 9, 2006

(54) Methods for identifying anatomical and molecular targets for analgesic therapy

(75) Inventors: David Borsook, Concord, MA (US); Lino Becerra, Cambridge, MA (US)

Correspondence Address: CLARK & ELBING LLP, 101 FEDERAL STREET, BOSTON, MA, 02110, US

(73) Assignee: The Mclean Hospital Corporation, Belmont (US)

(21) Appl. No.: 10/562,583

(22) PCT Filed: Jul. 9, 2004
(86) PCT No.: PCT/US04/22239

§ 371 Date: May 23, 2006

(30) Foreign Application Priority Data
Jul. 11, 2003 (US) 60486401

(51) Int. Cl. A61B 5/05

(52) U.S. Cl. 600/410

(57) ABSTRACT

This invention features methods for identifying targets for analgesic therapy by comparing the results of functional magnetic resonance imaging in non-human test subjects having genetically-based differences in their neural responses to painful stimuli.

### BACKGROUND OF THE INVENTION

Functional magnetic resonance imaging (fMRI) is a non-invasive in vivo imaging technique that allows a clinician to monitor brain activity with high spatial and temporal resolution. Functional MRI is based on the increased local blood flow resulting from neural activity and the concomitant reduction in deoxyhemoglobin. Because deoxyhemoglobin is paramagnetic, changes in the local concentration alter the T2-weighted MRI signal and the effect is detectable using a standard clinical (1.5 T) MRI scanner.

Functional MRI provides several advantages over other in vivo imaging techniques (i.e., positron emission tomography (PET) and single photon emission tomography (SPECT)). Notably, fMRI does not require the use of radioisotopes and the data acquisition time is relatively short (2-4 minutes per scan). Additionally, high resolution images, with a pixel size of less than 1 mm, are possible.

The sensation of pain is universal, affecting every person in some manner. Pain can range in intensity and duration and frequently results from a variety of traumatic or pathologic conditions. Idiopathic pain is also common. While significant strides have been made in pain management, it is clear that our understanding of pain and nociceptive mechanisms is incomplete. Accordingly, there is a need to identify and characterize the anatomical, neurochemical, and genetic substrates of the central pain pathways.

### SUMMARY OF THE INVENTION

The present invention is based on the discovery that anatomical and molecular targets for analgesic therapy can be identified in the central nervous system by comparing fMRI results of individuals having genetic-based differences in their response to pain and analgesia. The methodology can also be adapted for use as a drug discovery tool, as well as a clinical tool useful for customizing analgesic therapy for individual patients. The comparison of central nociceptive responses between genetically diverse individuals improves on traditional methodologies that compare genetically similar individuals because it enables the detection of alternate central pain processing and analgesic pathways, as well as dominant pathways, facilitating the generation of more complete and more accurate brain maps for both pain perception and analgesic effects. The anatomic and mechanistic insights provided by interstrain comparisons using non-human subjects are also applicable to the natural diversity of responses observed in human populations, allowing for screens in animals that are reproducible over interrupted assay schedules. The fMRI brain mapping techniques of this invention also enable the predication of analgesic efficacy by allowing brain activation and suppression patterns (i.e., brain maps) triggered by a test compound alone to be compared to brain maps of known analgesics and brain maps of subjects administered specific types of painful stimuli. Further, the present invention may be used in unanesthetized subjects, enabling the detection of low level brain activation changes that would normally be obscured or altered by anesthetic administration.

Accordingly, in one aspect, the present invention features a method for identifying targets for analgesic therapy by providing two non-human test subjects having a genetic-based difference in nociception, performing an fMRI on the brains of the subjects during or following administration of a painful stimulus, and comparing the results of the fMRIs to identify one or more brain regions that are differentially activated in response to the painful stimulus. The identified brain regions are targets for analgesic therapy.

Optionally, the test subjects may be administered an analgesic, and a second fMRI is performed on the brains of the subjects during or following a painful stimulus, and the results compared to identify a brain region(s) that is differentially activated in response to the painful stimulus in the presence of the analgesic. The analgesic may be administered prior to, simultaneous with, or following the painful stimulus. The first and second fMRI scans of are compared within and between test subjects. Brain regions that are not modulated in the same manner, by anesthetic administration, in the presence and absence of the painful stimulus, between subjects are identified as targets for analgesic therapy.

In one embodiment of either method, the method comprises the further step of assessing gene expression in the identified, target brain region in order to identify a gene or gene product that is differentially expressed between the two subjects. A differentially expressed gene or gene product is a target for analgesic therapy.

In another aspect, the invention features a method for identifying targets for analgesic therapy by providing two non-human test subjects having a genetic-based difference in nociception, performing an fMRI on the brains of the subjects following administration of an analgesic, and comparing the results of the fMRIs to identify one or more brain regions that are differentially activated in response to the painful stimulus. The identified brain regions are targets for analgesic therapy.

In one embodiment, the method comprises the further step of assessing gene expression in the identified, target brain region in order to identify a gene or gene product that is differentially expressed between the two subjects. A differentially expressed gene or gene product is a target for analgesic therapy.

In preferred embodiments of any of the foregoing methods, the non-human subjects are rodents, more preferably, mice. Most preferably, one or more of the subjects is a 129P3, A, AKR, BALB/c, C3H/He, C57BL/6, C57BL/10, C58, CBA, DBA/2, RIIIS, SM, LP, SJL, or SWR mouse. Preferably, these strains are from the Jackson Laboratories breeding colony (Bar Harbor, Me.). In another preferred embodiment, the subjects are conscious (unanesthetized).

In another aspect, the invention features is a method for identifying targets for analgesic therapy using transgenic non-human test subjects. Specifically, this method provides two test subjects, one test subject expressing a transgene of interest and one not. An fMRI is performed on the brains of each of the subjects in the presence of a painful stimulus and the results compared. Non-identical results indicate that the gene of interest, or its product, is a target for analgesic therapy. Desirably, the gene of interest is expressed in the central nervous system. More desirably, the gene of interest is a human gene. Desirably, the non-human test subjects are rodents (e.g, rats or mice). Optionally, an analgesic is administered prior to, simultaneous with, or subsequent to the painful stimulus.

In another aspect, the invention features a method for identifying targets for analgesic therapy by providing two non-human test subjects, one subject having a gene of interest functionally disrupted or deleted. An fMRI is performed on the brains of each of the subjects in the presence of a painful stimulus and the results compared. Non-identical results indicate that the gene of interest, or its product, is a target for analgesic therapy. Desirably, both endogenous alleles of the gene of interest are functionally disrupted or deleted. Desirably, the non-human test subjects are rodents (e.g, rats or mice). Optionally, an analgesic is administered prior to, simultaneous with, or subsequent to the painful stimulus.

In the two preceding aspects, optionally, the test subjects may be administered an analgesic, and a second fMRI is performed on the brains of the subjects during or following a painful stimulus, and the results compared to identify a brain region(s) that is differentially activated in response to the painful stimulus in the presence of the analgesic. The analgesic may be administered prior to, simultaneous with, or following the painful stimulus. The first and second fMRI scans of are compared within and between test subjects. Brain regions that are not modulated in the same manner, by anesthetic administration, in the presence and absence of the painful stimulus, between subjects are identified as targets for analgesic therapy.

In another aspect, the invention features is a method for identifying targets for analgesic therapy using transgenic non-human test subjects. Specifically, this method provides two test subjects, one test subject expressing a transgene of interest and one not. An fMRI is performed on the brains of each of the subjects following the administration of an analgesic and the results compared. Non-identical results indicate that the gene of interest, or its product, is a target for, or modulates, analgesic therapy. Desirably, the gene of interest is expressed in the central nervous system. More desirably, the gene of interest is a human gene. Desirably, the non-human test subjects are rodents (e.g, rats or mice). In one embodiment, a painful stimulus may be administered to the test subject prior to or during the fMRI scan. In another embodiment, a painful stimulus is administered to the subject subsequent the first fMRI scan, but during the therapeutically effective period of analgesia, and a second fMRI scan is performed. The first and second fMRI scans of are compared within and between test subjects. Brain regions that are not modulated in the same manner, by anesthetic administration, in the presence and absence of the painful stimulus, between subjects are identified as targets for analgesic therapy.

In another aspect, the invention features a method for identifying targets for analgesic therapy by providing two non-human test subjects, one subject having a gene of interest functionally disrupted or deleted. An fMRI is performed on the brains of each of the subjects following the administration of an analgesic and the results compared. Non-identical results indicate that the gene of interest, or its product, is a target for analgesic therapy. Desirably, both endogenous alleles of the gene of interest are functionally disrupted or deleted. Desirably, the non-human test subjects are rodents (e.g, rats or mice). In one embodiment, a painful stimulus may be administered to the test subject prior to or during the fMRI scan. In another embodiment, a painful stimulus is administered to the subject subsequent the first fMRI scan, but during the therapeutically effective period of analgesia, and a second fMRI scan is performed. The first and second fMRI scans of are compared within and between test subjects. Brain regions that are not modulated in the same manner, by anesthetic administration, in the presence and absence of the painful stimulus, between subjects are identified as targets for analgesic therapy.

Suitable painful stimuli for use in any of the foregoing methods include, for example, conditions that cause acute, chronic, neuropathic, arthritic, or cancer-induced pain. The painful stimulus may be mechanical, chemical, or thermal and may be administered to a naïve subject or one that has been pre-treated in order to induce a hypersensitivity reaction.

Any analgesic suitable for treating acute or chronic pain or any candidate compound whose analgesic effects are under investigation may be used in the foregoing methods. Suitable analgesics include, but are not limited to, channel blockers (e.g., calcium, sodium, potassium, and chloride channel blockers), antidepressants, anti-seizure medications, μ-opioid receptor agonists, κ-opioid receptor agonists, cannabinoid receptor agonists, nicotinic receptor agonists, glutamatergic receptor antagonists (e.g., NMDA receptor and AMPA receptor antagonists), GABA receptor agonists (e.g, benzodiazepines), dopaminergic receptor agonists, and adrenergic receptor agonists. Other suitable analgesics include membrane stabilizers such as local anesthetics. In particular, useful analgesics include, for example, gabapentin, lamotrignine, ketamine, amitriptyline, clonazepam, diazepam, and morphine.

As used herein, by “target for analgesic therapy” is meant any brain region, neuronal pathway, or molecular (e.g., RNA and protein) target that influences nociception or analgesia. Anatomical targets for analgesic therapy include, for example, brain regions involved in sensory evaluation of pain (e.g., dorsal horn, dorsal column nuclei, thalamus, and primary somatosensory cortex); brain regions involved in emotional/motivational response to pain (e.g., nucleus accumbens, sublenticular extended amygdala, hippocampus, amygdala, anterior cingulated cortex, and orbitofrontal cortex), brain regions involved in autonomic response to pain (e.g., hypothalamus), and brain regions involved in endogenous modulation (inhibition or facilitation) of pain (e.g., periaqueductal gray). Molecular targets for analgesic therapy including, for example, proteins and nucleic acids that encode neurotransmitter receptors (e.g., glutamatergic, serotonergic, dopaminergic, adrenergic, cholinergic, and GABAergic receptors), enzymes (e.g., tyrosine hydroxylase, choline acetyl transferase, and acetyl cholinesterase), or other proteins in the intraneuronal signal transduction pathways that are normally activated in response to a painful stimulus. Other suitable targets include transcription factors normally activated or inhibited in response to a painful stimulus.

By “nociception” is meant the sensation of pain.

By “acute pain” is meant a sensation of pain directly attributable to a stimulus. Acute pain ceases at the same time or substantially the same time as the painful stimulus.

By “chronic pain,” when used in connection with a painful stimulus of defined intensity and duration, is meant a sensation of pain that persists for some time after the removal/cessation of the stimulus. “Chronic pain” may also be used in connection with the sensation of pain caused by disease (e.g., arthritis or cancer). This type of chronic pain is persistent in the absence of a stimulus (other than the disease state).

By “hypersensitive pain” is meant a disproportionately intense sensation of pain in response to a stimulus. The stimulus may be of sufficient intensity to illicit nociception in a normal subject, or the stimulus may be below the normal threshold for nociception. Hypersensitive pain is distinguishable from chronic pain because, for hypersensitive pain, no pain sensation exists in the absence of a stimulus. Nociceptive hypersensitivity may occur following an acutely painful stimulus, but after the acute pain phase has subsided.

By “painful stimulus” is meant any stimulus that induces nociception. When referring to acute pain, a pain stimulus reversibly induces nociception with a duration limited to the application of the stimulus and a short time thereafter. For example, nociception following chemical-induced acute pain (i.e., formalin or capsaicin models described below) lasts for minutes or hours after application whereas, nociception induced by thermal or physical (e.g., von Frey filaments) stimuli does not persist substantially beyond the stimulus duration. By contrast, chronic or neuropathic pain are persistent pain states. As such, the painful stimulus is often administered well in advance of the fMRI studies such that the test subject is sensing pain at the time of the study.

### DETAILED DESCRIPTION

The present invention is based on the discovery that anatomical and molecular targets for analgesic therapy can be identified by comparing functional MR scans of individuals having genetic-based differences in their response to pain and analgesia. This strategy may be applied to human and non-human subjects alike and, in the case of human subjects, may be used to customize individual analgesic therapy. Further, using the present methodology, brain responses to pain may be measured in unanesthetized subjects and, in a single session, a variety of nociceptive conditions (i.e., pain stimulus alone, analgesic treatment alone, and the combination of a painful stimulus and an analgesic) may be tested.

This methodology provides several advantages over traditional studies of brain responses to pain. Previous fMRI studies attempted to compare brain responses between individuals having highly similar, or even identical, genetic backgrounds. While this strategy may be useful for probing targets or mechanisms that are identified a priori, it provides little or no insight into the mechanisms underlying the range of responses to pain and analgesia frequently observed in populations having diverse genetic backgrounds (e.g., human populations). It is expected that the brains of genetically similar individuals process painful sensations and respond to analgesics using similar brain structures and anatomical pathways. Thus, studies of genetically diverse individuals allow for the discovery of alternate pain pathways within the brain, leading to novel targets for analgesic therapy.

In addition The fMRI method described below permits brain imaging at a time simultaneous to a painful stimulus or administration of an analgesic in unanesthetized subjects. The use of unanesthetized subjects is desirable because it allows the detection of low-level activation of ancillary brain structures that might normally be masked or confounded by anesthesia.

Another advantage of the present invention is that it allows for the creation of a library of fMRI brain maps from subjects of diverse genetic backgrounds (i) in the presence of various types of painful stimuli, (ii) following the administration of various analgesics, and (iii) following the administration of various analgesics and in the presence of a painful stimulus. This library can be used, for example, to identify and develop novel analgesics by comparing the effect of a test compound with the effect of one or more known analgesics as a predictor of the analgesic potential of the test compound. Further, these brain maps may be used to customize analgesic therapy in human patients. It is expected that different patients will centrally process painful sensations differently. With a brain map of the patient experiencing pain, analgesic therapy can be tailored using drugs which most specifically and most effectively target the identified brain regions. Customized analgesic therapy also affords the opportunity to treat refractory pain and/or to reduce the incidence of adverse effects (e.g., nausea and addiction).

Finally, the fMRI methodology of this invention can be used in conjunction with standard molecular biological assays, in non-human subjects, to identify novel molecular targets for analgesic therapy. For example, when comparing subjects having different genetic backgrounds and different central responses to pain, the differentially activated brain regions may be microdissected and assayed for differences in gene expression or protein levels.

The present invention therefore features methods for identifying anatomical and molecular targets for analgesic therapy useful in diagnosis, treatment, and drug discovery. The method involves performing functional magnetic resonance imaging (fMRI) on human or non-human test subjects having genetically-based differences in their responses to pain or analgesia and identifying brain regions that are differentially activated in the presence of pain, following the administration of an analgesic (or test compound), or both. In addition to identifying target brain regions and pathways, molecular targets can also be assessed. Genetically-based differences in nociception and analgesia can also be investigated using transgenic and/or knockout animals. Preferably, these animals have an identical genetic background, with the exception of the inserted or deleted gene. In this way, the investigator is able to attribute differences in brain activity, detected using fMRI, to the presence or absence of one specific gene product.

Whether comparing among individual non-human test subjects of different strains, or between transgenic or knock-out animals and wild-type littermates, the fMRI scans may be performed (i) in the presence of a painful stimulus, (ii) following administration of an analgesic, or (iii) in the presence of a painful stimulus and following administration of an analgesic. Optionally, test subjects may be pre-treated in such a manner as to induce hypersensitivity or allodynia.

Test Subjects

Test subjects useful in the methods of this invention include any individuals that have been characterized as having differences in nociception or responses to analgesia. Suitable non-human test subjects include, for example, non-human primates and rodents (e.g., rats and mice). Mice are particularly useful test subjects because of the relative ease in experimentation and the relatively complete understanding of the murine genome. Recent studies have demonstrated a strong correlation among nociceptive tests of similar modalities (Lariviere et al., Pain 97: 75-86, 2002). For example, similar results are obtained in tests of mechanical hypersensitivity in the von Frey monofilament tests whether dynorphin or peripheral nerve ligation is used as the sensitizing treatment. Animals differing in nociception can be readily identified using any standard assay for pain response (for example, those provided below), and identifying animals (i.e., of different strains) that exhibit differences in pain response, analgesic response, or both. Table 1 outlines some examples of interstrain differences in measured responses to various nociceptive stimuli that have been identified among a variety of inbred murine strains.

TABLE 1
Marine Strain Differences in Response to Nociceptive Stimuli

| Nociceptive Stimulus | Strong Responders | Weak Responders |
|---|---|---|
| Thermal Nociception | 129P3, A, AKR, BALB/c, | C57BL/6, C57BL/10, |
| | C3H/He, RIIIS | C58 |
| Chemical Nociception | AKR, C3H/He, C57BL/6, C58, | 129P3, SM |
| (Bee venom) | CBA, DBA/2, RIIIS | |
| Chemical Nociception | C3H/He, C57BL/6, C57BL/10, | 129P3, AKR, BALB/c |
| (Capsaicin) | C58, CBA, DBA/2 | |
| Thermal Hypersensitivity | 129P3, CBA | A, AKR, C3H/He, |
| | | DBA/2 |
| Mechanical Hypersensitivity | 129P3 | C57BL/10 |
| Afferent-dependent | C3H/He, CBA, DBA/2, SM | C57BL/10, BALB/c |
| Hypersensitivity | | |

These mice provide particularly useful test subjects for the methods of the invention.

Other useful test subjects include any animal into which a transgene may be introduced. Such transgenic technology is standard in the art and may be applied to the generation of desired rodents (such as mice and rats) as well as larger animals. Preferably, the introduced transgene (which may be of human origin) is related to neuronal function.

Other useful test subjects include knock-out animals, which may also be used to identify putative targets for analgesic therapy. An animal, such as a mouse, that has had one (knock-down) or both alleles (knock-out) of a target gene inactivated (e.g., by homologous recombination or by insertional mutagenesis) is a preferred animal model for screening, identifying, and/or confirming the effect of a putative target on nociception. This approach enables targeted changes to be made to a gene, such that the altered gene can be passed from one generation to the next. Knock-outs may be generated, for example, by the use of mouse embryonic stem (ES) cells. These cells may be genetically modified in vitro and then implanted into a foster mother, where they develop into embryos and are brought to term. The resulting offspring are derived from the altered ES cells and carry the introduced genetic modification in their genome. Since mice, like humans, contain two copies of every gene, one from each parent, the first generation of mice reared from the modified ES cells contains one copy of the mutant gene and one healthy variety. A single round of interbreeding leads to mice with two copies of the mutant gene and the full manifestation of the introduced mutation; alternatively, mice born by foster mothers are bred with wild type mice to produce heterozygotes, and these heterozygotes are interbred to produce knock-out mice.

Instead of mutating or deleting a polynucleotide sequence from the genome of a non-human subject, it may instead be desirable to insert a polynucleotide sequence into the genome. This technique, commonly referred to as “knocking in,” can be accomplished using many of the methods described for the production of knock-out mice. In some instances, it may be desirable to “knock in” a putative target for analgesic therapy in order to replace the polynucleotide encoding the orthologous mouse target. The knocked-in polynucleotide may be expressed under the control of the endogenous mouse regulatory sequence, or may have exogenous regulatory sequences.

Knock-out and knock-in mice are produced according to methods well known in the art (see, e.g., Manipulating the Mouse Embryo. A Laboratory Manual, 2nd ed. B. Hogan, R. Beddington, F. Constantini, E. Lacy, Cold Spring Harbor Laboratory press, 1984). For any of these approaches, inbred strains of genetically identical mice are of immense value as starting animals for test subject production. The uniformity of these mice provides repeatable results, particularly in studies, such as fMRI studies, in which measurements from control and test conditions must be measured in different animals.

Painful Stimuli

At least five dissociable types of nociception and hypersensitivity exist in mice. They include (i) thermal nociception, (ii) noxious chemical nociception, (iii) thermal hypersensitivity, (iv) mechanical hypersensitivity, and (v) afferent input-dependent hypersensitivity (Lariviere et al., Pain 97: 75-86, 2002). Many suitable models exist for mimicking and assaying each category of nociception and hypersensitivity. Exemplary models are provided below but are not intended to be limiting.

Thermal Nociception

Any standard tail-withdrawal test may be used for measuring either thermal nociception or thermal hypersensitivity. In one particular method, subjects are restrained for fMRI scanning. During the scan, their tail is placed in a water bath maintained at, for example, 49.0±0.2° C. The latency to tail removal from the water bath, usually by a strong dorsal tail flexion, is measured. Unlike other thermal nociceptive tests (i.e., the hot-plate test), this test is amenable to repeated measurements and adaptable to simultaneous fMRI scanning. This test may be modified to measure thermal nociception to cold stimuli by replacing the water with −15° C. ethanol. These conditions are exemplary and may be easily modified, as necessary, to accommodate assays involving different strains, species, or other experimental variables.

Noxious Chemical Nociception

Spontaneous pain can be induced by an intraplantar injection (about 25-50 μl per injection) of a noxious chemical substance including, for example, capsaicin, formalin, carageenen or bee venom. In one model, about 0.2 mg of honey bee (Apis mellifera) venom (Sigma Chemical Co., St. Louis, Mo., catalog #3375) is administered in a single injection. Symptoms include edema, spontaneous elevation, licking, and shaking of the injected paw. The spontaneous pain response typically peaks at 5-10 minutes post-injection and lasts for up to one hour.

In other models of spontaneous chemical-induced nociception, a 5% solution of formalin (37% w/w formaldehyde) or a solution containing 2.5 μg capsaicin are injected into the plantar hindpaw. The 5% formalin model typically results in a biphasic response having an acute nociceptive phase during the first 10 minutes post injection, and a delayed inflammatory phase lasting about 10-60 minutes post injection.

Abdominal constriction (writhing) tests are also useful for measuring acute nociception in response to noxious chemicals. In these tests, chemicals such as acetic acid, acetylcholine, adenosine triphosphates, bradykinin, hypertonic saline, magnesium sulfate, or phenylquinone are injected into the peritoneal cavity where they activate nociceptors directly and/or produce inflammation of visceral and subcutaneous tissues (see, for example, Mogil et al., Pain 80:67-82, 1999, and references therein). The most commonly used methodology for inducing abdominal writhing is a single intraperitoneal injection of either 0.6% glacial acetic acid or 120 mg/kg magnesium sulfate in a volume of 10 ml/kg. The onset of acetic acid-induced nociception is about five minutes after injection and lasts for about 30 minutes. Magnesium sulfate-induce nociception has a virtually immediate onset but lasts for only about five minutes.

Thermal Hypersensitivity

Thermal hypersensitivity may be induced either in an acute model of hyperalgesia or following nerve injury. An acute model may be induced, for example, by a intraplantar injection of 50 μl of 2% carrageenan. The hypersensitivity effect peaks about 3-5 hours post injection. Carrageenan, unlike bee venom, does not usually induce a significant acute nociceptive effect and therefore eliminates the potentially confounding effect of measuring both acute noxious chemical noinception and thermal hypersensitivity.

Thermal hypersensitivity following carrageenan injection may be measured using a modification of the Hargreaves' test. Normally, the Hargreaves' test subjects are placed in cages having a glass floor (usually 3/16-inch) and allowed to acclimate until they become inactive. The floor is heated from below by aiming an incandescent light bulb (45 W, 6 cm below the floor) at the hindpaw of an inactive subject and paw withdrawal latency is measured. This test is readily adaptable to restrained subjects during fMRI testing. A distal extension of the hindlimb exposes the plantar hindpaw, allowing for a focused thermal stimulus to be directed at the hindpaw. In addition to the fMRI scan, hindpaw withdrawal latency can also be measured.

Mechanical Hypersensitivity

Dynamic or static mechanical nociception may be measured using a brush test or a modified von Frey filament test respectively. Measurement of dynamic mechanical nociception is performed using camel hair brushes or other similar substance (e.g., Velcro and cloth). The stimulus is applied at 1-2 Hz and the time for paw withdrawal is measured. For static mechanical hyperalgesia, subjects are placed on a metal mesh floor and the foot withdrawal latency is measured following stimulation with various calibrated von Frey monofilaments having bending forces ranging from 0.3 to 53.9 mN which are administered in a single and steady application of greater than one second (see, for example, Chaplan et al., J. Neurosci. Meth. 53: 553-63, 1994).

These tests may be modified for subjects restrained in an fMRI scanner by a distal extension of the hindlimb, exposing the plantar hindpaw. The hindlimb is held lightly when extended and the withdrawal latency is measured upon a direct application of the von Frey monofilament. Using this test, it is possible to detect either increased sensitivity (hindlimb withdrawal following stimulation using a lower bending force (thinner) filament) or decreased sensitivity (hindlimb withdrawal following stimulation using a higher bending force (thicker) filament).

Afferent Input-Mediated Hypersensitivity

Afferent input mediated hypersensitivity may be present following acute pain or chronic/neuropathic pain. Neuropathic pain may be produced by a number of methods including, for example, neurotoxin treatment (e.g., vincristine), surgical methods, cytokine application to the nerve (e.g., TNF-alpha), endocrine disruption (e.g., streptozotocin treatment). Neuropathic pain produced by surgical methods involve damage to a nerve (usually the sciatic or femoral nerves or their branches). Useful models include, for example, (i) the spinal nerve ligation (SNI) model (Kim et al. Pain 50: 355-363, 1992), (ii) the sciatic nerve ligation (SNL) model (Decosterd et al. Pain 87:149-158, 2000), and (iii) the chronic constriction injury (CCI) model (Bennett et al. Pain 33:87-107, 1988).

Spinal Nerve Ligation (SNL): Rats are anesthetized, placed in a prone position, and a L4 to S2 midline incision made. The paraspinal muscles are separated from the spinous processes at these levels, followed by identification and ligation of the left L5 and L6 spinal nerves using 6-0 silk thread.

Chronic constriction injury (CCI): Under anesthesia, the common sciatic nerve is exposed and loose constrictive ligatures are placed above the trifurcation.

Spared nerve injury (SNI): Rats are anesthetized, the trifurcation in exposed and the sciatic nerve and its 3 main branches (sural, common peroneal, and tibial nerves) are identified. The common peroneal and tibial nerves are cut and ligated. Typically, this is a unilateral procedure.

Cold Allodynia Model: Neuropathic pain is induced in awake animals on an elevated grid by gently apply a drop of acetone to the lateral plantar skin of the paw using a blunt needle. Cooling is produced by the acetone evaporation. The duration of paw withdrawal is recorded with an arbitrary maximum cut off of 20 sec.

Toxin Models: Afferent input-mediated hypersensitivity may be measured during the delayed hypersensitivity phase following an intraplantar injection of about 0.2 mg of honey bee (Apis mellifera) venom (Sigma Chemical Co., St. Louis, Mo., catalog #3375). Venom treatment results in spontaneous and acute pain, lasting for up to one hour followed by a hypersensitivity phase lasting for about 7-8 hours. To avoid the confounding effects of the spontaneous chemical nociception phase, afferent input-dependent hypersensitivity using the modified Hargreaves' test is usually measured 2-4 hours post-injection. Preliminary studies may be performed to optimize the venom dose and define the duration of the acute nociceptive versus hypersensitivity phases of the response.

Depending on the afferent input-mediated hypersensitivity model, but beginning as early as the fourth day post-surgery, fMRI scanning and nociceptive testing using either by application of thermal, mechanical or chemical stimuli is performed.

Analgesic Treatment

Although the clinician currently has a wide array of analgesics affecting diverse mechanisms from inflammatory processes to neurotransmitter receptor transmission, a complete understanding of the intracellular effects of these compounds, particularly in central neurons, is lacking. Accordingly, it is desirable to perform fMRI screening and gene expression studies on test subjects in the presence of a painful stimulus, following analgesic treatment, and/or in the presence of a painful stimulus and analgesic treatment. These approaches provide a more complete understanding of the signal transduction mechanisms and the changes in gene expression that accompany both the painful stimuli and analgesic treatment, enabling more specific and effective analgesics to be developed.

Desirably, analgesic therapy is administered, in the presence or absence of a painful stimulus, at therapeutically relevant doses. Suitable analgesics include, for example, μ-opioid receptor agonists such as morphine (5-200 mg/kg), κ-opioid receptor agonists such as U50,488 (10-150 mg/kg), cannabinoid receptor agonists such as WIN 55,212-2 (0.5-480 μg/kg), nicotinic receptor agonists such as epibatidine (7.5-150 μg/kg), adrenergic receptor agonists such as clonidine (0.1-5.0 mg/kg), membrane stabilizers (e.g, local anesthetics), anti-seizure medications such as gabapentin and lamotrigine, NMDA receptor antagonists such as ketamine, antidepressants such as amitriptyline, and benzodiazepines such as diazepam and clonazepam.

Useful doses of any suitable analgesic can be readily determined prior to experimentation and will depend upon the genetic background (strain) of the test subject as well as the pain modality being tested.

Some genetic-based differences in analgesic responses are understood. For example, differences in morphine antinociception between mouse strains have been mapped to several “quantitative trait loci” (QTLs) which account for most of the variability. In the case of morphine, the QTLs encompass genes encoding the μ-opioid receptor and the serotonin-1B receptor (Belknap et al., Life Sci. 57: P117-P124, 1995; Hain et al., J. Pharmacol. Exp. Ther. 291: 444-449, 1999). More recently, Wilson et al. (J. Pharmacol. Exp. Ther. 304: 547-559, 2003) have demonstrated that the antinociceptive effects of different classes of analgesics vary in potency by as much as 37-fold among different inbred mouse strains (Table 2).

TABLE 2
Murine Strain Differences in Response to Different Classes of Analgesics

| | Sensitive Strains | Resistant Stains |
|---|---|---|
| Analgesic Class | (low AD₅₀) | (high AD₅₀) |
| μ-opioid receptor agonist | 129P3, C3H/He, DBA/2 | C58 |
| (morphine) | | |
| κ-opioid receptor agonist | 129P3, BALB/c, DBA/2, RIIIS | C57/BL6, C58, CBA |
| (U50, 488) | | |
| Cannabinoid receptor agonist | 129P3, A, DBA/2 | AKR, C57BL/10, C58 |
| (WIN 55, 212-2) | | |
| Nicotinic receptor agonist | A, BALB/c | AKR, CBA, C57BL/10 |
| (Epibetadine) | | |
| Adrenergic receptor agonist | 129P3, A, AKR, | C57BL/10, C57BL/6, |
| (Clonidine) | | C58 |

AD₅₀ = the half-maximal antinociceptive dose.

Functional Magnetic Resonance Imaging

As indicated above, fMRI is performed on test subjects of the invention following administration of a painful stimulus, analgesic treatment, or both, to identify brain regions that are differentially activated. Preferably, the fMRI is performed in animals that are awake and adequately restrained, although, anesthetized animals can also be used. One particularly useful apparatus for restraining awake animals for fMRI is described in detail in U.S. Pat. No. 6,275,723, hereby incorporated by reference. Briefly, the animal is gently restrained by insertion into a body tube with its head protruding through a circular aperture that is the head holder. The head holder also has a bite bar and a vertical nose clamp to secure the head onto the bite bar. The animal's skull is further stabilized by lateral ear clamping screws, attached to the frame of the restraining device, which are inserted into the auditory canal of the animal. Animals may be lightly anesthetized (i.e., 300 mg/kg chloral hydrate) to facilitate their positioning in the restraining device. Desirably, the animals fully recover from anesthesia prior to applying the painful stimulus and MR imaging.

Magnetic resonance images may be acquired, for example, using a Bruker BioSpec 70/20AS spectrometer (Bruker BioSpin, Billerica, Mass.). A set of images prior to the fMRI scans may be acquired to shim the magnetic field, optimize receiver gains and RF pulse lengths. Similar images may be collected to obtain anatomical reference slices using a RARE 256×256×24 prescription with 0.117×0.117×1.0 mm voxel size. Such images may be used to co-register functional and anatomical images to a standard atlas. Functional images (sensitive to the blood oxygen level ) are T2-weighted and acquired using a FSE-RARE sequence of a 12 slice-prescription with a FOV of 30 mm and 1.5 mm slice-thickness, a matrix size of 64×64, echo trail length of 16 and an effective TE of 53 ms. Image reconstruction and processing is typically carried off-line using appropriate image analysis software such as MEDx (Sensor Systems, Bethesda Md.).

A multi subject statistical analysis maybe used to obtain differential activation across regions of interest (ROI). Depending on the nature of the comparison between groups a random or fixed effects model maybe used. Such comparisons maybe carried out on a voxel-by-voxel approach, on an ROI-based approach, or on a cluster-analysis approach. In a typical analysis, parametric statistics may be used.

Target Identification

Targets for analgesic therapy identified by the methods of this invention can be brain regions, neuronal pathways, or molecular targets. Comparison of functional MR results of brain activation patterns in response to a painful stimulus among individual test subjects having genetic differences in nociception is used to identify differences in the central pain processing pathways. Intrastrain comparisons reveal that a different connectivity of central pathways exists in sensitive strains of animals. This difference in connectivity may be observed, for example, as a supranormal activation or suppression, in response to pain, of a brain region in one strain, compared to another. Altered nociception may, therefore, be attributed to heightened (or attenuated) activation of a particular brain region. Alternatively, altered pain sensitivity may be attributed to the activation (or suppression of a brain region not otherwise recognized as being involved in a nociceptive pathway. These studies may also be used to develop a library of brain maps of the response to various types of pain in each of the genetic backgrounds.

Functional MR images from test subjects administered an analgesic (or a test compound) in the presence or absence of pain are also used to reveal the analgesic's anatomical targets. Brain maps of activation and suppression patterns can be developed for use in conjunction with other studies. For example, comparing brain maps of candidate compounds to those of known analgesics is useful for predicting the analgesic effects of those candidate compounds. Furthermore, understanding the anatomical targets of test compounds aids in the development of drugs having a higher specificity for pain-mediating pathways and simultaneously reducing the addictive properties. For example, analgesics may be developed that activate/suppress brain regions involved in nociception such as the dorsal horn, dorsal column nuclei, thalamus, and primary somatosensory cortex (sensory evaluation of pain); the nucleus accumbens, sublenticular extended amygdala, hippocampus, amygdala, anterior cingulated cortex, and orbitofrontal cortex (emotional/motivational response to pain); the hypothalamus (autonomic response to pain); and the periaqueductal gray (modulation of pain), but not those brain regions that mediate addiction (e.g., the ventral tegmental area and the nucleus accumbens).

These pain and analgesia brain maps are also useful for predicting the effect of combination analgesic therapy. For example, an fMRI of a patient suffering from pain may reveal a brain activation pattern that is not effectively treatable using a single analgesic compound. An understanding of the neuronal effects of each analgesic compound allows for a specific combination therapy to be tailored for each patient where the drug combination targets only those brain regions necessary to effect analgesia in that particular patient. Similarly, an understanding of the brain regions targeted by each analgesic can be used to reduce double prescriptions by physicians. For example, it may be unnecessary to prescribe two different analgesic compounds if each of the compounds has a similar brain activation pattern. A better clinical outcome may result from the prescription of combinations of analgesics that result in complementary patterns of brain activation and suppression. In this way, more targeted analgesic therapy may be delivered while simultaneously reducing the incidence of adverse effects.

The fMRI studies described above may also be combined with molecular biological techniques which, in addition to characterizing anatomical targets, can be used to identify molecular targets of analgesic therapy. Specifically, gene expression studies (e.g., Northern blotting, Western blotting, and gene chip analysis) may be used to characterize the interstrain genetic differences underlying the variable responses to painful stimuli and analgesic therapies, as described further below.

Assessment of Gene Expression

Following identification of brain regions that are differentially activated, gene expression is assessed in these regions. Any number of methods are available for measuring such gene expression. For example, gene expression may be measured using microarray (gene chip) analysis as a rapid and inexpensive screen to identify novel targets. Methods for making and using nucleic acid microarrays are known to the skilled artisan and are described, for example, in U.S. Pat. No. 5,837,832, Lockhart et al. (Nat. Biotech. 14:1675-1680, 1996), and Schena et al. (Proc. Natl. Acad. Sci. 93:10614-10619, 1996), herein incorporated by reference. Methods for making and using polypeptide microarrays are described, for example, by Ge (Nucleic Acids Res. 28:e3.i-e3.vii, 2000), MacBeath et al. (Science 289:1760-1763, 2000), Zhu et al. (Nature Genet. 26:283-289), and in U.S. Pat. No. 6,436,665, hereby incorporated by reference.

Alternatively, if previously identified genes are under investigation, Northern blot analysis or RT-PCR may be performed, using any appropriate fragment prepared from the nucleic acid molecule as a hybridization probe or primer (see, for example, Ausubel et al., supra; PCR Technology: Principles and Applications for DNA Amplification, ed., H. A. Ehrlich, Stockton Press, NY; and Yap and McGee, Nucl. Acids Res. 19:4294, 1991).

Immunoassays may also be used to detect or monitor a candidate protein that may be an analgesic target. Specific polyclonal or monoclonal antibodies may be used in any standard immunoassay format (e.g., ELISA, Western blot, or RIA assay) to measure the protein levels. Examples of immunoassays are described, e.g., in Ausubel et al. (supra). Immunohistochemical techniques may also be used to visualize the anatomical and/or cellular localization of the protein of interest. For example, fixed post-mortem brains may be sectioned using a freezing microtome and stained for the presence of the candidate protein using an antibody against that protein and any standard detection system (e.g., one which includes a secondary antibody conjugated to horseradish peroxidase). General guidance regarding such techniques can be found in, e.g., Bancroft and Stevens (Theory and Practice of Histological Techniques, Churchill Livingstone, 1982) and Ausubel et al. (supra).

In any of the above methods, expression levels of the candidate gene or protein that correlate directly or inversely with differential activation levels in response to pain or analgesic treatment identify the candidate gene or gene product as a molecular target for analgesic therapy.

### Other Embodiments

All publications and patent applications cited in this specification are hereby incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for identifying a target for analgesic therapy, said method comprising the steps of:
(a) providing a first and a second non-human subject, wherein said subjects have a genetic-based difference in nociception;
(b) performing an fMRI on the brain of said first subject during or following administration of a painful stimulus;
(c) performing an fMRI on the brain of said second subject during or following administration of said painful stimulus; and
(d) comparing the results of said fMRI on the brain of said first subject with the results of said fMRI on the brain of said second subject to identify a brain region that is differentially activated in response to said painful stimulus, said brain region being a target for analgesic therapy.

2. The method of claim 1, wherein said method further comprises the steps of:
(e) administering an analgesic;
(f) performing a second fMRI on the brain of said first subject during or following a second administration of said painful stimulus;
(g) performing a second fMRI on the brain of said second subject during or following a second administration of said painful stimulus; and
(h) comparing the results of said second fMRIs to identify a brain region that is differentially activated in response to said painful stimulus in the presence of said analgesic, said brain region being a target for analgesic therapy.

3. The method of claim 1, wherein, prior to, simultaneous with, or following administration of said painful stimulus, an analgesic is administered to said first subject and said second subject and, in step (d), said brain region is differentially activated in response to said painful stimulus, said analgesic, or both.

4. The method of claim 1, wherein said method further comprises the step of:
(e) assessing gene expression in said target brain region identified in step (d) to further identify a gene or gene product that is differentially expressed, wherein said differentially expressed gene or gene product is a target for analgesic therapy.

5. The method of claim 2, wherein said method further comprises the step of:
(i) assessing gene expression in said target brain region identified in step (h) to further identify a gene or gene product that is differentially expressed, wherein said differentially expressed gene or gene product is a target for analgesic therapy.

6. The method of claim 1, wherein said first subject and said second subject are rodents.

7. The method of claim 6, wherein said rodents are of different strains.

8. The method of claim 6, wherein at least one of said rodents is selected from the group consisting of mice of the strains 129P3, A, AKR, BALB/c, C3H/He, C57BL/6, C57BL/10, C58, CBA, DBA/2, RIIIS, SM, LP, SJL, and SWR.

9. The method of claim 1, wherein said painful stimulus is an acute pain stimulus.

10. The method of claim 1, wherein said painful stimulus is a chronic pain stimulus.

11. The method of claim 10, wherein said chronic pain stimulus is neuropathic pain, arthritic pain, or cancer pain.

12. The method of claim 1, wherein said painful stimulus is a stimulus that induces a hypersensitive response.

13. The method of claim 1, wherein said first subject and said second subject are conscious.

14. A method for identifying a target for analgesic therapy, said method comprising the steps of:
(a) administering an analgesic to a first and a second non-human subject, wherein said subjects have a genetic-based difference in nociception;
(b) performing a first fMRI on the brain of said first subject during or following administration of said analgesic;
(c) performing a first fMRI on the brain of said second subject during or following administration of said analgesic; and
(d) comparing the results of said fMRI on the brain of said first subject with the results of said fMRI on the brain of said second subject to identify a brain region that is differentially activated in response to said analgesic administration, said brain region being a target for analgesic therapy.

15. The method of claim 14, wherein said method further comprises the step of:
(e) assessing gene expression in said target brain region identified in step (d) to further identify a gene or gene product that is differentially expressed, wherein said differentially expressed gene or gene product is a target for analgesic therapy.

16. The method of claim 14, wherein said first subject and said second subject are rodents.

17. The method of claim 16, wherein said rodents are of different strains.

18. The method of claim 16, wherein at least one of said rodents is selected from the group consisting of mice of the strains 129P3, A, AKR, BALB/c, C3H/He, C57BL/6, C57BL/10, C58, CBA, DBA/2, RIIIS, SM, LP, SJL, and SWR.

19. The method of claim 14, wherein said first subject and said second subject are conscious.

20. The method of claim 14, wherein said analgesic is a channel blocker, antidepressant, μ-opioid receptor agonist, κ-opioid receptor agonist, cannabinoid receptor agonist, nicotinic receptor agonist, or adrenergic receptor agonist.

21. The method of claim 14, wherein said analgesic is morphine.

22. A method for identifying a target for analgesic therapy, said method comprising the steps of:
(a) providing a first and a second non-human subject, said second non-human subject differing from said first non-human subject in its expression of a transgene of interest;
(b) performing an fMRI on the brain of said first subject during or following administration of said painful stimulus;
(c) performing an MRI on the brain of said second subject during or following administration of a painful stimulus; and
(d) comparing the results of said fMRI on the brain of said first subject with the results of said fMRI on the brain of said second subject, wherein non-identical results indicate that said transgene of interest or its product is a target for analgesic therapy.

23. The method of claim 22, wherein said method further comprises the steps of:
(e) administering an analgesic;
(f) performing a second fMRI on the brain of said first subject during or following a second administration of a painful stimulus;
(g) performing a second fMRI on the brain of said second subject during or following a second administration of a painful stimulus; and
(h) comparing the results of said second fMRIs to identify a brain region that is differentially activated in response to said painful stimulus in the presence of said analgesic, said brain region being a target for analgesic therapy.

24. The method of claim 22, wherein, prior to, simultaneous with, or following administration of said painful stimulus, an analgesic is administered to said first subject and said second subject and, in step (d), said brain region is differentially activated in response to said painful stimulus, said analgesic, or both.

25. The method of claim 22, wherein said transgene is expressed in the central nervous system.

26. The method of claim 22, wherein said subjects are rodents.

27. The method of claim 22, wherein said painful stimulus is an acute pain stimulus.

28. The method of claim 22, wherein said painful stimulus is a chronic pain stimulus.

29. The method of claim 28, wherein said chronic pain stimulus is neuropathic pain, arthritic pain, or cancer pain.

30. The method of claim 22, wherein said painful stimulus is a stimulus that induces a hypersensitive response.

31. The method of claim 22, wherein said first subject and said second subject are conscious.

32. A method for identifying a target for analgesic therapy, said method comprising the steps of:
(a) providing a first and a second non-human subject, said second non-human subject differing from said first non-human subject in that at least one endogenous allele of a gene of interest is functionally disrupted or deleted;
(b) performing an fMRI on the brain of said first subject during or following administration of a painful stimulus;
(c) performing an fMRI on the brain of said second subject during or following administration of said painful stimulus; and
(d) comparing the results of said fMRI on the brain of said first subject with the results of said fMRI on the brain of said second subject, wherein non-identical results indicate that said gene of interest or its product is a target for analgesic therapy.

33. The method of claim 32, wherein said method further comprises the steps of:
(e) administering an analgesic;
(f) performing a second MRI on the brain of said first subject during or following a second administration of said painful stimulus;
(g) performing a second fMRI on the brain of said second subject during or following a second administration of said painful stimulus; and
(h) comparing the results of said second fMRIs to identify a brain region that is differentially activated in response to said painful stimulus in the presence of said analgesic, said brain region being a target for analgesic therapy.

34. The method of claim 32, wherein, prior to, simultaneous with, or following administration of said painful stimulus, an analgesic is administered to said first subject and said second subject and, in step (d), said brain region is differentially activated in response to said painful stimulus, said analgesic, or both.

35. The method of claim 32, wherein, in said second non-human subject, both endogenous alleles of said gene of interest are functionally disrupted or deleted.

36. The method of claim 32, wherein said first subject and said second subject are rodents.

37. The method of claim 32, wherein said painful stimulus is an acute pain stimulus.

38. The method of claim 32, wherein said painful stimulus is a chronic pain stimulus.

39. The method of claim 38, wherein said chronic pain stimulus is neuropathic pain, arthritic pain, or cancer pain.

40. The method of claim 32, wherein said painful stimulus is a stimulus that induces a hypersensitive response.

41. The method of claim 32, wherein said first subject and said second subject are conscious.

42. A method for identifying a target for analgesic therapy, said method comprising the steps of:
(a) administering an analgesic to a first and a second non-human subject, said second non-human subject differing from said first non-human subject in its expression of a transgene of interest;
(b) performing an fMRI on the brain of said first subject following administration of said analgesic;
(c) performing an fMRI on the brain of said second subject following administration of said analgesic; and
(d) comparing the results of said fMRI on the brain of said first subject with the results of said fMRI on the brain of said second subject, wherein non-identical results indicate that said transgene of interest or its product is a target for analgesic therapy.

43. The method of claim 42, wherein said first subject and said second subject are rodents.

44. The method of claim 42, wherein said analgesic is a channel blocker, antidepressant, μ-opioid receptor agonist, κ-opioid receptor agonist, cannabinoid receptor agonist, nicotinic receptor agonist, or adrenergic receptor agonist.

45. The method of claim 42, wherein said analgesic is morphine.

46. The method of claim 42, wherein, prior to step (a), said first and said second non-human subjects are administered a painful stimulus.

47. The method of claim 42, wherein said first subject and said second subject are conscious.

48. A method for identifying a target for analgesic therapy, said method comprising the steps of:
(a) administering an analgesic to a first and a second non-human subject, said second non-human subject differing from said first non-human subject in that at least one endogenous allele of a gene of interest is functionally disrupted or deleted;
(b) performing an fMRI on the brain of said first subject following administration of said analgesic;
(c) performing an fMRI on the brain of said second subject following administration of said analgesic; and
(d) comparing the results of said fMRI on the brain of said first subject with the results of said fMRI on the brain of said second subject, wherein non-identical results indicate that said gene of interest or its product is a target for analgesic therapy.

49. The method of claim 48, wherein, in said second non-human subject, both endogenous alleles of said gene of interest are functionally disrupted or deleted.

50. The method of claim 48, wherein said first subject and said second subject are rodents.

51. The method of claim 48, wherein said analgesic is a channel blocker, antidepressant, μ-opioid receptor agonist, κ-opioid receptor agonist, cannabinoid receptor agonist, nicotinic receptor agonist, or adrenergic receptor agonist.

52. The method of claim 48, wherein said analgesic is morphine.

53. The method of claim 48, wherein, prior to step (a), said first and said second non-human subjects are administered a painful stimulus.

54. The method of claim 48, wherein said first subject and said second subject are conscious.
